(19) United States
(12) Patent Application Publication
(10) Pub. No.: US 2014/0134701 A1
(43) Pub. Date: May 15, 2014

(54) BACTERIORHODOPSIN-CONTAINING BIOACTIVE GEL AND METHOD FOR FABRICATING THE SAME

(71) Applicant: B.R.L. LABORATORY INC., Taipei (TW)

(72) Inventor: Alexander BRASLAVSKIY, Taipei (TW)

(73) Assignee: B.R.L. LABORATORY INC., Taipei (TW)

(21) Appl. No.: 13/728,670

(22) Filed: Dec. 27, 2012

(30) Foreign Application Priority Data
Nov. 9, 2012 (TW) 101141769

(51) Int. Cl. C12N 11/04

(52) U.S. Cl. CPC C12N 11/04
USPC 435/178; 435/182

(57) ABSTRACT

The present invention discloses a bacteriorhodopsin-containing bioactive gel and a method for fabricating the same, wherein a thickening polymeric compound is suspended in water to form a base gel having appropriate glutinosity and suitable for skin, and wherein a bacteriorhodopsin-containing culture fluid, which is obtained via culturing halobacteria in a spent culture fluid, is integrated with the glutinous base gel to form the bacteriorhodopsin-containing bioactive gel. The bioactive gel of the present invention is abundant of vitamins, minerals, amino acids, etc. and thus very useful in beauty treatment and dermatological treatment. The present invention is also expected to have high commercial potential in many other fields.

### BACKGROUND OF THE INVENTION

1. Field of the Invention

The present invention relates to bacteriorhodopsin, particularly to a bacteriorhodopsin-containing bioactive gel and a method for fabricating the same.

2. Description of the Related Art

Scientists pay attention to halobacteria and the like because their purple membrane contains a protein similar to rhodopsin, i.e. visual purple, which is a biological pigment in photoreceptor cells of the retina that is responsible for the first events in the perception of light. As rhodopsin is the only protein in the purple membrane of halobacteria, it is also called the bacteriorhodopsin. Bacteriorhodopsin can function as a unidirectional light-driven proton pump. Bacteriorhodopsin can harness light energy to synthesize ATP (adenosine triphosphate), which is a process similar to photosynthesis. In a dark environment, bacteriorhodopsin can undertake oxidative phosphorylation to facilitate bacteria growth. Bacteriorhodopsin in the purple membrane has special structure and function and is popularly regarded as the simplest biomolecule able to convert light energy in addition to chlorophyl of green plants. Therefore, bacteriorhodopsin is deemed to be one of the most promising photoelectric materials for green energy. Bacteriorhodopsin may be applied to many fields, including artificial retinae, 3D optical memories, optical switches, photoelectric sensors, image edge detectors, monochromatic filters for optical images, anti-fake materials, biochips, bio-hydrogen energy systems, fuel cells, etc.

The Inventors have been devoted to researching everything about halobacteria persistently. From studying the bacteriorhodopsin generated by halobacteria and developing the applications thereof, the Inventors learned that the culture fluid generating bacteriorhodopsin is abundant of vitamins, minerals, and amino acids, etc. and should be very useful in beauty treatment. The culture fluid generating bacteriorhodopsin is also expected to have high commercial potential in many other fields.

### SUMMARY OF THE INVENTION

The primary objective of the present invention is to provide a bacteriorhodopsin-containing bioactive gel and a method for fabricating the same, wherein a bacteriorhodopsin-containing culture fluid is integrated with a glutinous base gel to achieve bioactive effects, including promoting skin quality and treating some dermatoses, wherefrom many people will be benefited.

To achieve the abovementioned objective, the present invention proposes a bacteriorhodopsin-containing bioactive gel, which comprises a base gel and a bacteriorhodopsin-containing culture fluid, wherein the base gel is formed via mixing a thickening polymeric compound with water, and wherein the bacteriorhodopsin-containing culture fluid is dispersed in the base gel, and wherein the bacteriorhodopsin-containing culture fluid has bacteriorhodopsin that is generated via culturing halobacteria in a spent culture fluid.

The present invention also proposes a method for fabricating a bacteriorhodopsin-containing bioactive gel, which comprises steps: mixing a thickening polymeric compound with water to obtain a glutinous base gel; and adding to the base gel a bacteriorhodopsin-containing culture fluid that is generated via culturing halobacteria in a spent culture fluid, to obtain the bacteriorhodopsin-containing bioactive gel of the present invention.

Bacteriorhodopsin-containing culture fluid has many nourishing components, such as vitamins, minerals, amino acids, etc., not only sustaining and reviving skin but also preventing and improving dermatoses. In some embodiments of the present invention, the abovementioned polymeric compound is the Carbopol gum or the Xanthan gum. In one embodiment, the Xanthan gum cooperates with an aqueous gum, such as the Carob gum, to maintain the bacteriorhodopsin-containing bioactive gel in appropriate glutinosity and make the bacteriorhodopsin-containing bioactive gel have a dosage form suitable for skin. The method of the present invention can fast fabricate the bacteriorhodopsin-containing bioactive gel. The bacteriorhodopsin-containing bioactive gel of the present invention can be spread on skin easily and uniformly, without blocking the pores. Other ingredients, such as essences or hydrophilic drugs, can be added to the bacteriorhodopsin-containing bioactive gel, contributing fine features and satisfying users' requirements.

The bacteriorhodopsin-containing bioactive gel of the present invention is expected to be a breakthrough medicosmetic product. Further, the present invention has expandability to develop other functions to meet the requirements of users. Therefore, the present invention has very high commercial potential.

Below, embodiments are described in detail in cooperation with the attached drawings to make easily understood the objects, technical contents, characteristics, and accomplishments of the present invention.

### BRIEF DESCRIPTION OF THE DRAWINGS

FIG. 1 is a flowchart of a method for fabricating a bacteriorhodopsin-containing bioactive gel according to one embodiment of the present invention.

### DETAILED DESCRIPTION OF THE INVENTION

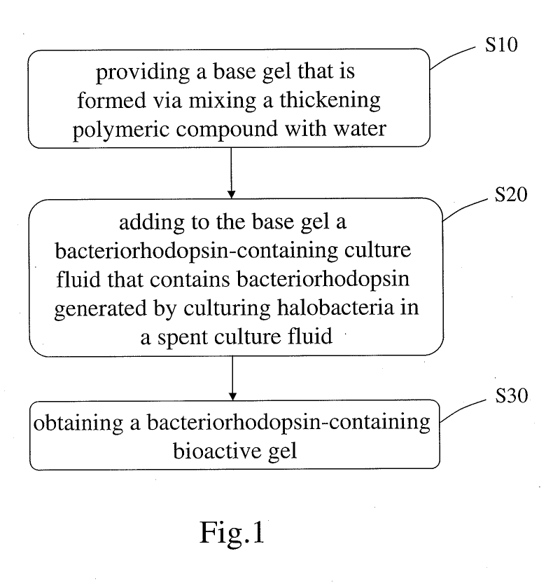

Refer to FIG. 1 a flowchart of a method for fabricating a bacteriorhodopsin-containing bioactive gel according to one embodiment of the present invention. In Step S10, provide a base gel, which is formed via suspending a thickening polymeric compound in water. In one embodiment, the abovementioned polymeric compound is the Carbopol gum or the Xanthan gum. Below are described the processes of using the two abovementioned polymeric compounds to fabricate the base gel.

(1) The process of using the Carbopol gum to fabricate the base gel:
a. Weighing the Carbopol gum, wherein the proportion of the Carbopol gum is 0.2-0.5 wt % in the base gel, i.e. each 100 g of the base gel has 0.2-0.5 g of the Carbopol gum;
b. Screening the Carbopol gum, adding the screened Carbopol gum into cool water having a low pH value, and placing the solution still for about 1 day to let the Carbopol gum absorb water and swell;
c. Homogenizing the solution for 30 minutes;
d. Undertaking a neutralization step on the solution, wherein a neutralizing agent is added to the solution with continuous agitation to modify the solution to have a pH value of 6-7, whereby is obtained a high-glutinosity and high-workability transparent gel, i.e. the base gel of the present invention.

The abovementioned neutralizing agent is selected from a group consisting of alkalis, soda, carbonates, ammonium hydrogen carbonate, ammonia, triethanolamine, NaOH, and KOH.

In one embodiment, triethanolamine is used as the neutralizing agent, and the ratio of the Carbopol gum and the triethanolamine is 1.0:1.5. In one embodiment, a hydroxide (sodium hydroxide or potassium hydroxide) is used as the neutralizing agent, and the ratio of the Carbopol gum and sodium hydroxide (or potassium hydroxide) is 1.0:2.3.

The abovementioned neutralization step is a critical one. The Carbopol gum has a carboxyl group (—COOH), which is likely to ionize and makes the aqueous solution of the Carbopol gum have a pH value of 3-4. As soon as the aqueous solution of the Carbopol gum has a pH value greater than 6, the polymeric molecular chains of the Carbopol gum, which are originally tangled with each other irregularly and tightly, are electrified to repel mutually. Thus, the molecular chains are dispersed and then interlocked to form a network. Thereby is increased the glutinosity of the solution of the Carbopol gum. In the neutralization step, the amount of the neutralizing agent added to the solution is much smaller than the amount of water in the solution. Therefore, the solution can be thickened to form a glutinous gel.

(2) The process of using the Xanthan gum to fabricate the base gel:
a. Weighing the Xanthan gum, wherein the proportion of the Xanthan gum is about 0.5-2.0 wt % in the base gel; (A low concentration of the Xanthan gum is sufficient to generate a very glutinous gel.)
b. Heating the solution to a temperature of 36-38° C.; (The Xanthan gum can dissolve well in hot water and cool water. However, it dissolves faster in the abovementioned temperature range.)
c. Making the Xanthan gum easy to agitate uniformly and hard to agglomerate into lumps in water, and preferably making the Xanthan gum easy to disperse in water; (To achieve that, the Xanthan gum should be thoroughly mixed with other ingredients. The abovementioned ingredient may be a dried material, such as cornstarch, clay or oat flour; then, water is added to the mixture. The abovementioned ingredient may be a non-aqueous solvent, such as glycerol, alcohol or vegetable oil. The Xanthan gum is mixed with the non-aqueous solvent, and only a few drops of the non-aqueous solvent are sufficient. The mixture is placed still for one or two minutes; then, the mixture is agitated uniformly.)
d. Adding water to the mixture containing the Xanthan gum, and agitating the mixture intensely to obtain the base gel.

In one embodiment, additional ingredients are added to the base gel, wherein the additional ingredient is added to water to form a solution firstly, and then the Xanthan gum is added to the solution. In one embodiment, a preservative is added to the base gel for preserving the product. In one embodiment, another aqueous gum, such as the Carob gum, is used together with the Xanthan gum.

After the abovementioned procedures is obtained the base gel. In one embodiment, an essence is added to the base gel to provide a pleasant smell. In one embodiment, a hydrophilic drug is added to the base gel to meet requirements of users.

Next, the process proceeds to Step S20. In Step S20, integrate the base gel with a bacteriorhodopsin-containing culture fluid. For fabricating the bacteriorhodopsin-containing culture fluid, halobacteria are cultured in a spent culture fluid to generate bacteriorhodopsin. The fluid containing bacteriorhodopsin is exactly the desired bacteriorhodopsin-containing culture fluid.

In an application of the present invention to treat psoriasis, a 25% sodium chloride solution is used as the spent culture fluid to culture halobacteria and generate bacteriorhodopsin. (In practice, a solution containing 26-31% sodium chloride can be also used as the spent culture fluid.) The bacteriorhodopsin-containing culture fluid has viable cells by a concentration of 0.3-4.0 g/L. Next, the process proceeds to Step S30. In Step S30, pour the bacteriorhodopsin-containing culture fluid and the base gel into a container, and mix them for 5 minutes to make the color uniform. Thus is completed the fabrication of the bacteriorhodopsin-containing bioactive gel of the present invention.

Tables. 1-4 are the results of the chromatographic analyses of the bacteriorhodopsin-containing culture fluid according to embodiments of the present invention.

TABLE 1
Amino acids

| Amino Acid | Content (mg/l) |
|---|---|
| aspartic acid | 134.4 |
| glutamic acid | 2.2 |
| serine | 27.3 |
| histidine | 120.4 |
| glycine | 101.6 |
| threonine | 15.7 |
| arginine | 15.5 |
| alanine | 220.3 |
| tyrosine | 103.4 |
| valine | 136.8 |
| methionine | 19.7 |
| tryptophan | 23.0 |
| phenylalanine | 37.9 |
| isoleucine | 35.7 |
| leucine | 58.2 |
| lysine | 414.3 |
| proline | 61.3 |

TABLE 2
Active metal cations

| Cation | Content (mg/l) |
|---|---|
| Li⁺ | N/A |
| NH₄⁺ | N/A |
| K⁺ | 42.81 |
| Ca²⁺ | 6.85 |
| mg²⁺ | 21.72 |

TABLE 3
Trace elements (the maximum contents)

| Trace Element | Contents (mg/l) |
|---|---|
| Cu | 0.009 |
| Fe | 0.191 |
| Mn | 0.0165 |
| P | 59.5 |
| S | 80.36 |
| Zn | 0.411 |

TABLE 4
Vitamins (the maximum contents)

| Vitamin | Content (mg/l) |
|---|---|
| B7(H) | 2.398 |
| choline | 2665.4 |
| chloride | |
| B12 | 0.1742 |
| folic acid | 146.665 |
| B8 | 7365 |
| B3(PP) | 3161 |
| B5 | 659.35 |
| B10 | 211.45 |
| B6 | 185.585 |
| B2 | 738.7 |
| B1 | 42.2 |

In conclusion, the bacteriorhodopsin-containing bioactive gel disclosed by the present invention has various nourishing components, including vitamins, minerals and amino acids, which are very helpful for sustaining skin health, reviving skin and treating some dermatoses. Therefore, the bioactive gel of the present invention can be used in medicosmetic products, etc. and will benefit many people.

The bacteriorhodopsin-containing bioactive gel of the present invention is fabricated into a dosage form having appropriate glutinosity and suitable for skin. Therefore, the bioactive gel of the present invention can be spread on skin fast and uniformly. Further, the bioactive gel of the present invention is easy to fabricate and would not block the pores. The bioactive gel of the present invention may further contain other components, such as essences or hydrophilic drugs, to present product features and satisfy customers.

It is expected that the bacteriorhodopsin-containing bioactive gel of the present invention will become a breakthrough medicosmetic product in the near future. The bioactive gel of the present invention has expandability to develop other functions to meet the requirements of users. Therefore, the present invention has very high commercial potential.

The present invention has been demonstrated with the embodiments described above. However, these embodiments are only to exemplify the present invention but not to limit the scope of the present invention. Any equivalent modification or variation according to the characteristic or spirit of the present invention is to be also included within the scope of the present invention, which is based on the claims stated below.

What is claimed is:

1. A bacteriorhodopsin-containing bioactive gel comprising
a base gel formed via mixing a thickening polymeric compound with water; and
a bacteriorhodopsin-containing culture fluid dispersed in said base gel, wherein said bacteriorhodopsin-containing culture fluid contains bacteriorhodopsin generated by culturing halobacteria in a spent culture fluid.

2. The bacteriorhodopsin-containing bioactive gel according to claim 1, wherein said polymeric compound is a Carbopol gum.

3. The bacteriorhodopsin-containing bioactive gel according to claim 2, wherein said base gel contains 0.2-0.5 wt % of said Carbopol gum.

4. The bacteriorhodopsin-containing bioactive gel according to claim 2, wherein said base gel is neutralized with a neutralizing agent selected from a group consisting of alkalis, soda, carbonates, ammonium hydrogen carbonate, ammonia, triethanolamine, NaOH, and KOH.

5. The bacteriorhodopsin-containing bioactive gel according to claim 4, wherein said triethanolamine is used as said neutralizing agent, and wherein a ratio of said Carbopol gum to said triethanolamine is 1.0:1.5.

6. The bacteriorhodopsin-containing bioactive gel according to claim 4, wherein said NaOH or said KOH is used as said neutralizing agent, and wherein a ratio of said NaOH or said KOH to said triethanolamine is 1.0:2.3.

7. The bacteriorhodopsin-containing bioactive gel according to claim 1, wherein said polymeric compound is a Xanthan gum.

8. The bacteriorhodopsin-containing bioactive gel according to claim 7, wherein said base gel contains 0.5-2.0 wt % of said Xanthan gum.

9. The bacteriorhodopsin-containing bioactive gel according to claim 7, wherein said base gel further comprises a dried component selected from a group consisting of cornstarch, clay and oat flour, and wherein said dried component is mixed with said Xanthan gum.

10. The bacteriorhodopsin-containing bioactive gel according to claim 7, wherein said base gel further comprises a non-aqueous solvent selected from a group consisting of glycerol, alcohol and vegetable oil, and wherein said non-aqueous solvent is mixed with said Xanthan gum.

11. The bacteriorhodopsin-containing bioactive gel according to claim 7, wherein said polymeric compound is mixed with a Carob gum.

12. The bacteriorhodopsin-containing bioactive gel according to claim 1, wherein said spent culture fluid is a solution containing 26-31% sodium chloride.

13. The bacteriorhodopsin-containing bioactive gel according to claim 1, wherein said bacteriorhodopsin-containing culture fluid is maintained to have 0.3-4.0 g of viable cells each liter.

14. A method for fabricating a bacteriorhodopsin-containing bioactive gel, comprising steps:
providing a base gel, wherein said base gel is formed via mixing a thickening polymeric compound with water; and
adding a bacteriorhodopsin-containing culture fluid to said base gel, wherein said bacteriorhodopsin-containing culture fluid contains bacteriorhodopsin generated by culturing halobacteria in a spent culture fluid.

15. The method for fabricating a bacteriorhodopsin-containing bioactive gel according to claim 14, wherein said polymeric compound is a Carbopol gum.

16. The method for fabricating a bacteriorhodopsin-containing bioactive gel according to claim 15, wherein said base gel contains 0.2-0.5 wt % of said Carbopol gum.

17. The method for fabricating a bacteriorhodopsin-containing bioactive gel according to claim 15, wherein after said Carbopol gum is mixed with water, a neutralizing agent is used to undertake a neutralization step, and wherein said neutralizing agent is selected from a group selected from a group consisting of alkalis, soda, carbonates, ammonium hydrogen carbonate, ammonia, triethanolamine, NaOH, and KOH.

18. The method for fabricating a bacteriorhodopsin-containing bioactive gel according to claim 17, wherein said triethanolamine is used as said neutralizing agent, and wherein a ratio of said Carbopol gum to said triethanolamine is 1.0:1.5.

19. The method for fabricating a bacteriorhodopsin-containing bioactive gel according to claim 17, wherein said NaOH or said KOH is used as said neutralizing agent, and wherein a ratio of said Carbopol gum to said NaOH or said KOH is 1.0:2.3.

20. The method for fabricating a bacteriorhodopsin-containing bioactive gel according to claim 14, wherein said polymeric compound is a Xanthan gum.

21. The method for fabricating a bacteriorhodopsin-containing bioactive gel according to claim 20, wherein said base gel contains 0.5-2.0 wt % of said Xanthan gum.

22. The method for fabricating a bacteriorhodopsin-containing bioactive gel according to claim 20, wherein in mixing said Xanthan gum with water, water is heated to a temperature of 36-38° C. to make said Xanthan gum dissolve faster.

23. The method for fabricating a bacteriorhodopsin-containing bioactive gel according to claim 20, wherein before said Xanthan gum is mixed with water, said Xanthan gum is mixed with a dried component selected from a group consisting of cornstarch, clay and oat flour.

24. The method for fabricating a bacteriorhodopsin-containing bioactive gel according to claim 20, wherein before said Xanthan gum is mixed with water, said Xanthan gum is mixed with a non-aqueous solvent selected from a group consisting of glycerol, alcohol and vegetable oil.

25. The method for fabricating a bacteriorhodopsin-containing bioactive gel according to claim 20, wherein said polymeric compound is mixed with a Carob gum.

26. The method for fabricating a bacteriorhodopsin-containing bioactive gel according to claim 14, wherein said spent culture fluid is a solution containing 26-31% sodium chloride.

27. The method for fabricating a bacteriorhodopsin-containing bioactive gel according to claim 14, wherein after culturing said halobacteria, said bacteriorhodopsin-containing culture fluid is maintained to have 0.3-4.0 g of viable cells each liter.
